(19) United States
(12) Patent Application Publication
(10) Pub. No.: US 2008/0311594 A1
(43) Pub. Date: Dec. 18, 2008

(54) USE OF FIBROBLAST GROWTH FACTOR 7 (Fgf7) AND OF THE RECEPTOR Fgfr2b AS BIOMARKERS

(76) Inventors: Jens Hoffman, Muhlenbeck (DE); Daniel Korr, Berlin (DE); Jan Kunde, Berlin (DE); Annete Sommer, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/134,697

(22) Filed: Jun. 6, 2008

Related U.S. Application Data
(60) Provisional application No. 60/944,153, filed on Jun. 15, 2007.

(30) Foreign Application Priority Data
Jun. 8, 2007 (DE) 10 2007 026 877.9

(51) Int. Cl. G01N 33/574; C07K 14/50; C12Q 1/02; C12Q 1/00

(52) U.S. Cl. 435/7.23; 530/399; 435/29

(57) ABSTRACT

The use of fibroblast growth factor Fgf7 and of the corresponding receptor Fgfr2b as biomarker candidates for the progesterone receptor antagonist 11β-(4-acetylphenyl)-17B-hydroxy-17α-(1,1,2,2,2-pentafluoroethyl)estra-4,9-dien-3-one, also known under the designations ZK230211 or ZK-PRA, of the formula

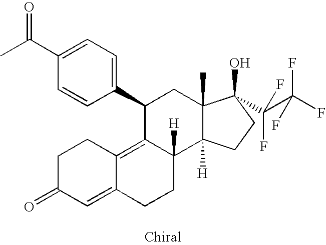

and for antiestrogens is described.

This application claims the benefit of the filing date of U.S. Provisional Application Ser. No. 60/944,153, filed Jun. 15, 2007.

The invention relates to the use of fibroblast growth factor Fgf7 and of the corresponding receptor Fgfr2b as biomarkers for the progesterone receptor antagonist 11β-(4-acetylphenyl)-17β-hydroxy-17β-(1,1,2,2,2-pentafluoroethyl)estra-4,9-dien-3-one and for antiestrogens.

The progesterone receptor antagonist 11β-(4-acetylphenyl)-17β-hydroxy-17α-(1,1,2,2,2-pentafluoroethyl)estra-4,9-dien-3-one, also known under the name ZK230211 or ZK-PRA, of the formula

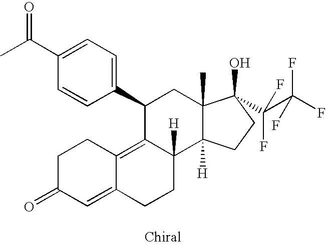

shows high antiprogestational activity together with few or no other endocrinological effects (Fuhrmann, U. et al., J. Med. Chem. 2000, 43, 5010-5016).

Fibroblast growth factor 7 (Fgf7) or else keratinocyte growth factor (kgf) belongs to a family of secreted glycoproteins which includes 22 members (Grose and Dickson 2005). Fgf7 is produced by cells of mesenchymal origin and binds specifically to the Fgfr2b which is expressed by epithelial cells. Fgf7 is thus a paracrine factor which mediates mesenchymal-epithelial signals. In xenograft experiments, MCF-7 tumours overexpressing Fgf7 were distinctly larger than tumours of MCF-7 cells without Fgf7 overexpression (Zang, Bullen et al. 2006). Fgf7 stimulates DNA synthesis, and proliferation and migration of tumour cells. Female rats to which Fgf7 was administered developed massive ductal hyperplasias, and mice overexpressing Fgf7 initially developed hyperplasias which developed into mammary carcinomas. Highly dedifferentiated mammary carcinomas without ER and PR showed very low levels of Fgfr2b, but well-differentiated tumours showed strong expression of Fgfr2b, so that it is assumed that the Fgf7-mediated stimulation and proliferation is a very early event in the molecular cascade which leads to progression and to metastasis. It has been shown in vitro that ER-positive cell lines such as MCF-7, T47-D and ZR75-1 show an increased proliferation and migration on stimulation with Fgf7, whereas ER-negative cell lines such as MDA-MB-231 do not (Zang and Pento 2000). It has been possible to show a correlation between the expression of Fgf7, Fgfr2b and the expression of ER-α. At the same time, the apoptosis rate in tumours with strong Fgf7 expression was distinctly lower (Tamaru, Hishikawa et al. 2004). The expression of Fgf7 in stromal cells is regulated by a large number of factors. It was possible to increase Fgf7 expression in fibroblasts by stimulation with the pro-inflammatory cytokines interleukin-1 and interleukin-6, which are produced by macrophages and some other cells. Other growth factors such as platelet-derived growth factor BB (pdgf BB) and transforming growth factor α likewise increased the expression of Fgf7 in mesenchymal cells (Finch and Rubin 2006).

It has also been shown that Fgf7 induces resistance of MCF-7 cells to treatment with tamoxifen. Addition of recombinant Fgf7 to the culture medium distinctly downregulated both the ER-α and PR at the mRNA and protein levels, so that tamoxifen showed no activity in proliferation tests. A positive feedback mechanism is postulated:

Fgf7 stimulates the production of endogenous aromatase, thus increasing the conversion of androgens into E2 (Chang, Sugimoto et al. 2006).

Fibroblast growth factor receptors (Fgfr) are transmembrane tyrosine kinases which are encoded by four structurally related genes (Fgfr1 to Fgfr4). Alternative splicing results in further isoforms of the receptors. The splice variants of Fgfr2 are Fgfr2b and Fgfr2c. Fgfr2b is produced only in cells of epithelial origin, and Fgfr2c only in mesenchymal cells. Fgfr2b is the specific receptor for the growth factor Fgf7 and is expressed in about 5% of breast tumours (Finch and Rubin 2006) and mediates signalling cascades via mitogen-activated protein kinase (MAPK) and phosphatidylinositol 3-kinase (PI3K) (Moffa, Tannheimer et al. 2004). In immunohistological investigations, FGFR2b was detected in the epithelial cells of breast tissues. A quantitative difference between normal tissue and malignant tissue was not detected in this case, and no FGFR2b was detectable in stromal cells (Palmieri, Roberts-Clark et al. 2003).

In view of the complex relationships of the cellular processes in the development of tumours and cancer and the control, connected therewith, of tumours and cancer, there remains a great need for the use of biomarkers with a view to treatment with active ingredients.

It has now surprisingly been found that an influence of treatment with 11β-(4-acetylphenyl)-17β-hydroxy-17α-(1,1,2,2,2-pentafluoroethyl)estra-4,9-dien-3-one on Fgfr2b gene expression is shown in cell lines.

Fgfr2b is the specific receptor for Fgf7.

It has also surprisingly been found that stimulation with Fgf7 correlates with development of resistance to treatment with 11β-(4-acetylphenyl)-17β-hydroxy-17α-(1,1,2,2,2-pentafluoroethyl)estra-4,9-dien-3-one (see T47D/T47D-resist). At the same time, cells with higher Fgfr2 expression respond less well or not at all to treatment with 11β-(4-acetylphenyl)-17β-hydroxy-17α-(1,1,2,2,2-pentafluoroethyl)-estra-4,9-dien-3-one, so that Fgfr2 is to be regarded as a potential stratifying marker for the use of 11β-(4-acetylphenyl)-17β-hydroxy-17α-(1,1,2,2,2-pentafluoroethyl)-estra-4,9-dien-3-one.

Thus, for example, it has been found that expression is higher in T47-D cells which are resistant to 11β-(4-acetylphenyl)-17β-hydroxy-17α-(1,1,2,2,2-pentafluoroethyl)-estra-4,9-dien-3-one than in sensitive cells.

Treatment with 11β-(4-acetylphenyl)-17β-hydroxy-17α-(1,1,2,2,2-pentafluoroethyl)-estra-4,9-dien-3-one leads to downregulation of Fgfr2 in the cells, pointing to the use of the receptor Fgfr2b as response marker. It has further been possible to show that reduction in FGFR2b expression in the resistant T47D cells by means of siRNA knock down restores the sensitivity to 11β-(4-acetylphenyl)-17β-hydroxy-17α-(1,1,2,2,2-pentafluoroethyl)estra-4,9-dien-3-one. This demonstrates that FGFR2b is a target for a combination therapy to overcome resistance or enhance the effect of 11β-(4-acetylphenyl)-17β-hydroxy-17α-(1,1,2,2,2-pentafluoroethyl)estra-4,9-dien-3-one by either small molecules which inhibit FGFR2b kinase, antibodies against FGFR2b or gene therapy.

The invention thus relates to the use of fibroblast growth factor Fgf7 as biomarker for the progesterone receptor antagonist 11β-(4-acetylphenyl)-17β-hydroxy-17α-(1,1,2,2,2-pentafluoroethyl)estra-4,9-dien-3-one

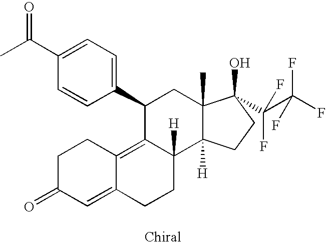

for the manufacture of a medicament for the treatment of cell growth associated with cancer and tumours.

The invention further relates to the use of the receptor Fgfr2b as biomarker for the progesterone receptor antagonist 11β-(4-acetylphenyl)-17β-hydroxy-17α-(1,1,2,2,2-pentafluoroethyl)estra-4,9-dien-3-one for the manufacture of a medicament for the treatment of cell growth associated with cancer and tumours, where the use of fibroblast growth factor Fgf7 or of the receptor Fgfr2b as stratifying marker for upregulation of Fgf7 in tumours or tumour cells and at high concentration of FGF7 in serum is associated with an intrinsic resistance to treatment with 11β-(4-acetylphenyl)-17β-hydroxy-17α-(1,1,2,2,2-pentafluoroethyl)estra-4,9-dien-3-one and a high Fgfr2 expression in the tumours and tumour cells.

The invention relates further to an in vitro method for determining the activity of the progesterone receptor antagonist 11β-(4-acetylphenyl)-17β-hydroxy-17α-(1,1,2,2,2-pentafluoroethyl)estra-4,9-dien-3-one in cell cultures, and in serum, where fibroblast growth factor Fgf7 or the receptor Fgfr2b is employed as biomarker for the determination.

In this connection, fibroblast growth factor Fgf7 or the receptor Fgfr2b are employed as stratifying markers for the upregulation of Fgf7 in tumours or tumour cells with resistance to treatment with 11β-(4-acetylphenyl)-17β-hydroxy-17α-(1,1,2,2,2-pentafluoroethyl)estra-4,9-dien-3-one or in cells with high Fgfr2 expression.

In their property as stratifying marker, the fibroblast growth factor Fgf7 and the receptor Fgfr2b are also employed as target for resistance modulation on use of 11β-(4-acetylphenyl)-17β-hydroxy-17α-(1,1,2,2,2-pentafluoroethyl)estra-4,9-dien-3-one.

The invention further relates to the use of fibroblast growth factor Fgf7 or of the receptor Fgfr2b as stratifying marker for the upregulation of Fgf7 in tumour cells with resistance to treatment with antiestrogens and to an in vitro method in which fibroblast growth factor Fgf7 or the receptor Fgfr2b is employed as stratifying marker for upregulation of Fgf7 in tumour cells with resistance to treatment with antiestrogens.

Suitable antiestrogens which are employed together with fibroblast growth factor Fgf7 or with the receptor Fgfr2b are for example tamoxifen, raloxifene, droloxifen, toremifen, lasofoxifen, arzoxifen, GW5638*), EM-800**), idoxifen and basedoxifene.

*) The chemical structure is disclosed in Wilson et al., Endocrinology 138, 3901, (1997) and Wu et al., Mol. Cell, 18, 413, (2005).

**) The chemical structure is disclosed in Labrie et al., J. Steroid Biochem. Mol. Biol. 79, 213, (2001).

The present invention also relates to an in vitro imaging method for the non-invasive determination of FGF7 and FGFR2 in tumour tissue and tumour cells using antibodies against these proteins which comprise a label which permit imaging.

Labels which permit imaging are for example fluorescent labels or else radioactive labels.

Suitable fluorescent markers and suitable radioactive markers which can be used are generally known and sufficiently well described.

The invention further relates to an in vitro method for reducing the FGF7 expression of antisense, si RNA, sh RNA and ribozymes and for inactivating circulating FGF7-blocking antibodies and soluble receptors.

The following examples demonstrate the possibility of carrying out the invention without restricting the invention only to these examples.

### EXAMPLE 1

### Identification of tumours which respond well (responders) to treatment with 11β-(4-acetylphenyl)-17β-hydroxy-17α-(1,1,2,2,2-pentafluoroethyl)estra-4,9-dien-3-one, and tumours which continue to show good growth and indicate possible resistance mechanisms (non-responders) on treatment with 11β-(4-acetylphenyl)-17β-hydroxy-17α-(1,1,2,2,2-pentafluoroethyl)estra-4,9-dien-3-one

For the test, an MXT-M3 tumour which was no longer strictly hormone-dependent was transplanted into mice.

The MXT(+) tumour model was induced in C57BLxDBA2F1 mice by intraperitoneal administration of a urethane solution. The developing mammary tumour could be transplanted further to mice with the same genetic background (syngeneic) and is still an established model. The MXT(+) model is distinguished very particularly by the fact that the tumours express both ER and PR in physiological concentrations. Although there are other hormone receptor-positive tumour models, frequently ER and PR are not functional therein and show no translocation from the cytoplasm into the nucleus after stimulation. Nor is it possible to show growth inhibition by hormone ablation here. These models are unsuitable for investigating antihormonal substances (Watson, Medina et al. 1977).

The result of tumour growth is depicted in FIG. 1.

At the end of the test, the animals were sacrificed and the tumours were removed, and then the weight of the tumours was determined. The variation in the tumour weights is high in each group. The average tumour weights in the vehicle control group (427 mg) was 2.8 times higher than in the ovariectomized group (155 mg) and 2.2 times as high as in the group treated with 11β-(4-acetylphenyl)-17β-hydroxy-17α-(1,1,2,2,2-pentafluoroethyl)estra-4,9-dien-3-one (192 mg).

For further gene expression studies, the RNA of selected tumours was isolated and analysed further by GeneChip analyses and real-time PCR:

The five largest tumours from the vehicle control group were selected. It was intended thereby to ensure that genes found to be upregulated with the non-responders to 11β-(4-acetylphenyl)-17β-hydroxy-17α-(1,1,2,2,2-pentafluoroethyl)-estra-4,9-dien-3-one in the treatment group are not attributable to the exclusive growth in size of the respective tumour, but are upregulated specifically for tumour growth on exposure to the substance.

A limit was set in the ovariectomized group and that treated with 11β-(4-acetyl-phenyl)-17β-hydroxy-17α-(1,1,2,2,2-pentafluoroethyl)estra-4,9-dien-3-one: tumours having a weight higher than 100 mg were assessed as non-responders, and tumours with a weight less than 100 mg were classified as responders.

TABLE 1
Gene expression of Fgf7 and its receptor.
R = responders;
NR = non-responders

| | | Affymetrix | Real-time | Real-time |
|---|---|---|---|---|
| | | regulation | regulation | regulation |
| | Gene | ZK-PRA NR | ZK-PRA R | ZK-PRA NR |
| | sym- | versus | versus vehicle | versus vehicle |
| Gene name | bol | ZK-PRA R | control | control |
| Fibroblast growth | *Fgf7 | 2.30 (±0.33) | 0.69 (±0.38) | 3.12 (±0.68) |
| factor 7 | | | | |
| Fibroblast growth | Fgfr2 | — | 0.30 (±0.07) | 0.55 (±0.30) |
| factor receptor 2 | | | | |

Fgf7 is expressed 3.12 times more strongly in the non-responder tumours than in the vehicle controls, and there was a small downregulation in the responder tumours to 70% compared with the controls.

Fgfr2 is distinctly downregulated to 30% (responder tumours vs. control) and 55% in the non-responders vs. control, on treatment. Expression generally higher in non-responders however.

### EXAMPLE 2

### In Vivo Confirmation Experiment

It was possible in the investigation of the tumour RNA of the in vivo experiment from Example 1 to identify biomarker candidates. To confirm these potential candidates, an independent animal experiment was carried out with the same study design, increasing the number of animals from ten to 20 per group. At the end of the test, the tumours were removed, the tumour weight was determined and the tumours were stored at −80° C. until RNA was isolated. The results are depicted in FIG. 2/5.

In the confirmation experiment, the tumour weights showed high variability within the groups. The average tumour weight in the vehicle control group was 577 mg and was thus 3.2 times higher than in the ovariectomized group (180 mg) and 2.7 times as high as in the group treated with 11β-(4-acetylphenyl)-17β-hydroxy-17α-(1,1,2,2,2-pentafluoroethyl)estra-4,9-dien-3-one (215 mg). Exactly as in the experiment from Example 1, the average tumour weight of the group treated with 11β-(4-acetylphenyl)-17β-hydroxy-17α-(1,1,2,2,2-pentafluoroethyl)estra-4,9-dien-3-one was somewhat above that in the ovariectomized group, however distinctly below that in the control group.

In the preceding experiment, the average tumour weight of the vehicle control animals was not quite as much higher than that in the ovariectomized group and that treated with 11β-(4-acetylphenyl)-17β-hydroxy-17α-(1,1,2,2,2-pentafluoroethyl)estra-4,9-dien-3-one, suggesting a somewhat greater hormone dependence of the tumours in this experiment.

Exactly as in Test 1, the limit for response to the therapy and the non-response was set here at a tumour weight of 100 mg. The five largest tumours were selected from the control group. The RNA was isolated from the tumours, and gene expression of the biomarker candidates was determined.

The results are listed in the following table:

TABLE 1.2.2
Gene expression of Fgf7 and its receptor.
R = responders; NR = non-responders

| | | | Real-time |
|---|---|---|---|
| | | Real-time | regulation |
| | | regulation | ZK-PRA NR |
| | Gene | ZK-PRA R versus | versus |
| Gene name | symbol | vehicle control | vehicle control |
| Fibroblast growth | Fgf7 | 1.72 (±0.68) | 3.25 (±1.91) |
| factor 7 | | | |
| Fibroblast growth | Fgfr2 | 0.47 (±0.34) | 0.25 (±0.20) |
| factor receptor 2 | | | |

### EXAMPLE 3

### Western Blot Analysis of the Progesterone Receptor Under the Influence of FGF7

It is reported in the literature that FGF7 downregulates both ER and PR. In order to detect this effect, cells were incubated in medium with various amounts of FGF7 for 24 h and 72 h, and then the PR content was determined.

The influence of Fgf7 on the amount of PR is very clear. A distinct decrease in PR is seen with increase in concentration. Both isoforms, PR-A and PR-B, decrease greatly. The effect was unambiguous after incubation with Fgf7 both for 24 h and for 72 h.

The results are depicted in FIG. 3/5.

### EXAMPLE 4

### Cell Growth Under the Influence of FGF7

The influence of FGF7 on the activity of 11β-(4-acetylphenyl)-17β-hydroxy-17α-(1,1,2,2,2-pentafluoroethyl)estra-4,9-dien-3-one was investigated on T47-D cells. The cells showed a sensitive response to treatment with 11β-(4-acetylphenyl)-17β-hydroxy-17α-(1,1,2,2,2-pentafluoroethyl)estra-4,9-dien-3-one. 50 ng/ml FGF7 were added to the medium, and the activity of ZK-PRA was determined in a proliferation assay.

11β-(4-Acetylphenyl)-17β-hydroxy-17α-(1,1,2,2,2-pentafluoroethyl)estra-4,9-dien-3-one showed a good activity on the sensitive T47-D cells in the proliferation assay. The maximum growth control (medium with all additions and E2) was set correspondingly at 100%, and the minimum control (medium with all additions, but no E2 and stripped of serum) showed a growth of 42%. It was thus possible to inhibit growth by 58% through withdrawal of E2.

At the highest substance concentration (10⁻⁵ mol/l), the growth was inhibited by 42%, at 10⁻⁶ mol/l by 47% and at 10⁻⁷ mol/l by 50%. At lower concentrations there was a further increase in growth. The growth at the lowest concentration (10⁻¹¹ mol/l) was 114%.

On addition of FGF7 (50 ng/ml) to the medium, 11β-(4-acetylphenyl)-17β-hydroxy-17α-(1,1,2,2,2-pentafluoroethyl)estra-4,9-dien-3-one showed a smaller inhibition on the proliferation of the sensitive T47-D cells. In the minimum control (E2 ablation) it was possible to reduce cell growth by only 29%, and it was still 71% of that of the maximum control. At the highest concentration (10⁻⁵ mol/l), growth was inhibited by 24%, and by 18% at the next lower concentration. At the lowest concentration (10⁻¹¹ mol/l), growth reached 114% of the maximum control. The results are depicted in FIG. 4/5.

### EXAMPLE 5

### Relative Expression of Fgfr2 Versus 18S rRNA in 6 Model Cell Lines In Vivo

In the in vivo experiments (see above), expression of Fgfr2 was one half to one quarter less strong in the non-responder and in the responder tumours than in the untreated control group.

Expression of the receptor for fibroblast growth factor 7 (Fgf7) was not regulated in the sensitive T47-D cells on treatment with ZK-PRA (1.1 times higher on treatment with ZK-PRA). Expression in the ZK-PRA-resistant T47-D without treatment was twice as high as in the sensitive T47-D cells and declined somewhat with ZK-PRA treatment to 1.3 times the value of the untreated sensitive T47-D cells. Expression in all other cells was considerably less both in normal medium and on treatment with ZK-PRA:

The amount of mRNA in the untreated BT-474 cells was 21 times less than in the untreated T47-D cells and was reduced on treatment with ZK-PRA to a value of 37-times lower. The initial value found for MCF-7 was 64 times lower and with ZK-PRA treatment was in fact 153 times lower compared with untreated sensitive T47-D cells. Expression in ZR75-1 was 3.7 times lower than in untreated T47-D cells and was reduced with treatment to an expression which was 9.4 times lower compared with T47-D under normal conditions. Expression in MDA-MB 231 was in fact 10 000 times lower and was slightly above the limit of detection.

The results are depicted in FIG. 5/5.

It is evident from Example 1 for example that Fgf7 is expressed more than three times as strongly in the non-responder tumours than in the responder tumours compared with the vehicle control, and this was reproducible in the confirmation experiment.

No Fgf7 mRNA was detectable in human breast carcinoma cells in the in vitro experiments. Fgf7 is a growth factor which is produced in mesenchymal cells, so that no epithelial expression was to be expected for Fgf7 in the breast tumour cells. The fact that in vivo expression was found is related to the natural situation in the growth of tumours in animals, where the cells are surrounded by natural stromal cells which also grow into the tumour. Stromal cells include fibroblasts, endothelial cells, macrophages, mast cells and adipocytes. Higher expression of Fgf7 in the larger non-responder tumours (vs. responder tumours) because of the greater proportion of stroma can be ruled out because the analysed tumours of the vehicle control were in some cases three times as large, and Fgf7 expression in these tumours was three times lower, so that a specific effect can be assumed here.

The influence of the mesenchymal factor Fgf7 on the 11β-(4-acetylphenyl)-17β-hydroxy-17α-(1,1,2,2,2-pentafluoroethyl)estra-4,9-dien-3-one-sensitive cell line T47-D was investigated in vitro. It was possible to show that cells to whose medium recombinant Fgf7 was added markedly downregulate PR. The PR-A isoform of the receptor was downregulated so much that only a very weak band was evident on the Western blot, and thus the PR profile approximately corresponded to the PR-positive, but 11β-(4-acetylphenyl)-17β-hydroxy-17α-(1,1,2,2,2-pentafluoroethyl)estra-4,9-dien-3-one-resistant cell lines BT-474 and ZR75-1. It has been suggested that the effect of 11β-(4-acetylphenyl)-17β-hydroxy-17α-(1,1,2,2,2-pentafluoroethyl)estra-4,9-dien-3-one is mediated by the PR-A isoform.

Subsequently, the response of the sensitive T47-D cells to treatment with 11β-(4-acetylphenyl)-17β-hydroxy-17α-(1,1,2,2,2-pentafluoroethyl)estra-4,9-dien-3-one under the influence of Fgf7 was investigated. A proliferation assay without Fgf7 in the medium revealed a distinct dose-response relation of 11β-(4-acetylphenyl)-17β-hydroxy-17α-(1,1,2,2,2-pentafluoroethyl)estra-4,9-dien-3-one—growth inhibition was greater as the concentration increased, so that it was possible to reach 50% growth inhibition. No dose-response relation could be found on addition of Fgf7 (50 ng/ml) to the culture medium. The maximum growth inhibition was about 20%, but there was no correlation of dose and response. The effect could perhaps have been enhanced by higher concentrations of Fgf7; 500 ng/ml are employed in some cases in the literature, but preliminary experiments had shown that a concentration of 50 ng/ml brings about a marked downregulation of PR, which was no stronger with 100 ng/ml, so that this concentration was chosen.

It is of particular interest that the cells showed completely hormone-independent growth under the influence of Fgf7. This serves to demonstrate the importance of FGF7 as marker for resistance to antiestrogens which, after all, require ER as target.

T47-D cells proliferate only in the presence of E2, so that E2 ablation serves as check of maximum growth inhibition. In the proliferation test without Fgf7 it was thus possible to achieve a maximum inhibition of almost 60%. On addition of Fgf7 to the medium, the effect was substantially smaller and the inhibition was now only 29%. Fgf7 thus downregulates not only ER and PR, so that the target for antiestrogens and antiprogestagens is lost, but additionally acts per se as growth factor, so that antihormonal effects or ablation of hormones are compensated. Fgf7 is a potent biomarker candidate. It has been shown that Fgf7 in non-responder tumours on treatment with 11β-(4-acetylphenyl)-17β-hydroxy-17α-(1,1,2,2,2-pentafluoroethyl)estra-4,9-dien-3-one is upregulated three-fold compared with the control tumours in contrast to the responder tumours. This was confirmed in an independent, second in vivo experiment. Fgf7 is a unidirectional, paracrine factor which has a local effect, but is nevertheless secreted, so that investigation is required of whether an elevated Fgf7 serum level possibly correlates with a non-response to treatment with 11β-(4-acetylphenyl)-17β-hydroxy-17α-(1,1,2,2,2-pentafluoroethyl)-estra-4,9-dien-3-one. It has additionally been shown that the effect of 11β-(4-acetylphenyl)-17β-hydroxy-17α-(1,1,2,2,2-pentafluoroethyl)estra-4,9-dien-3-one was markedly reduced under the influence of Fgf7, and the hormone dependence of T47-D cells was almost abolished.

### EXPLANATION OF THE FIGURES

FIG. 1/5: shows the change in the tumour area in the MXT tumour model on treatment with 11β-(4-acetylphenyl)-17β-hydroxy-17α-(1,1,2,2,2-pentafluoroethyl)-estra-4,9-dien-3-one.

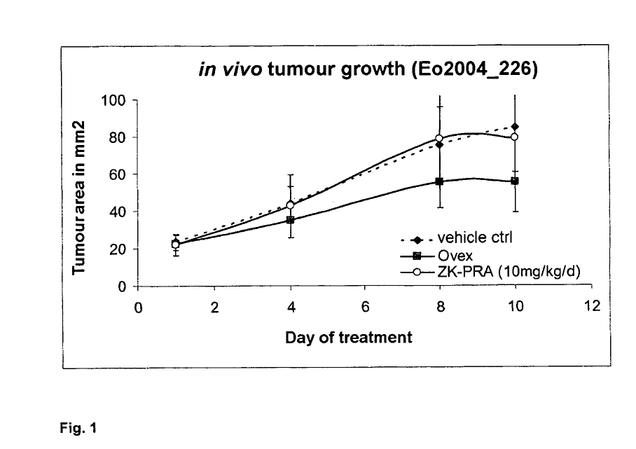

FIG. 2/5: shows the change in the tumour area in the MXT tumour model on treatment with 11β-(4-acetylphenyl)-17β-hydroxy-17α-(1,1,2,2,2-pentafluoroethyl)-estra-4,9-dien-3-one (test reproduction).

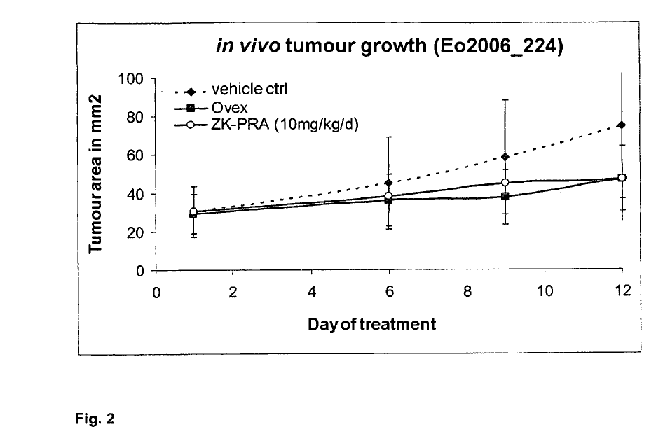

FIG. 3/5: shows T47-D cells which were cultured with addition of Fgf7 (20 ng/ml and 50 ng/ml) in the culture medium for 24 h and 72 h and from which the amount of PR was then determined in a Western blot.

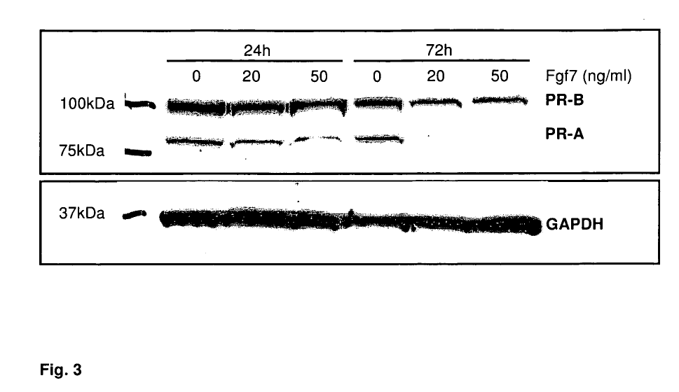

FIG. 4/5: shows the proliferation assay of T47-D cells with 11β-(4-acetylphenyl)-17β-hydroxy-17α-(1,1,2,2,2-pentafluoroethyl)estra-4,9-dien-3-one under the influence of Fgf7.

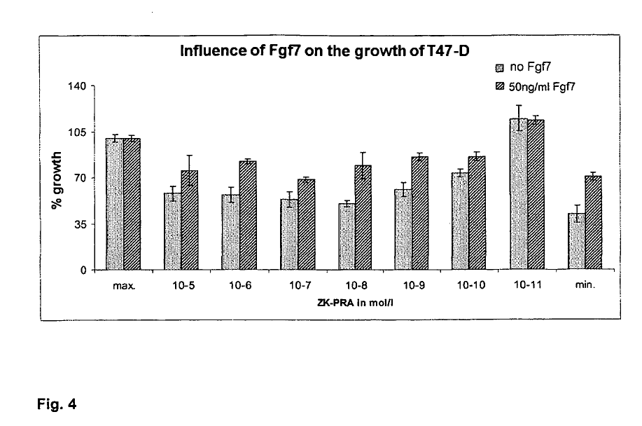

FIG. 5/5: shows the relative expression of Fgfr2 vs. 18S rRNA in 6 model cell lines.

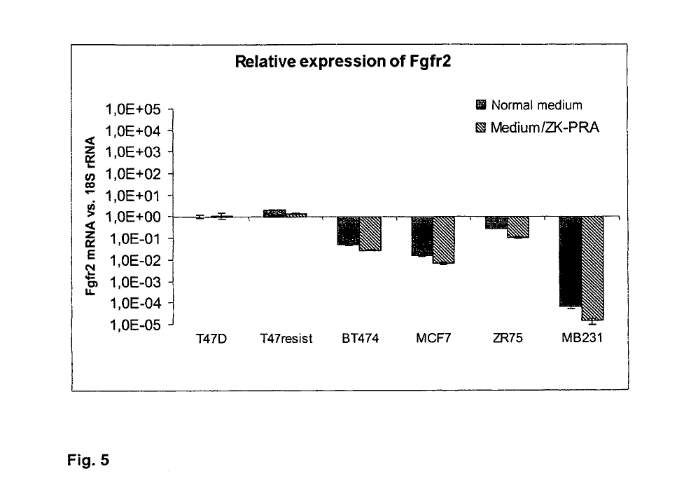

### REFERENCES

Chang, H. L., Y. Sugimoto, et al. (2006). “Keratinocyte growth factor (KGF) induces tamoxifen (Tam) resistance in human breast cancer MCF-7 cells.” Anticancer Res 26(3A): 1773-84.
Finch, P. W. and J. S. Rubin (2006). “Keratinocyte growth factor expression and activity in cancer: implications for use in patients with solid tumors.” J Natl Cancer Inst 98(12): 812-24.
Grose, R. and C. Dickson (2005). “Fibroblast growth factor signaling in tumorigenesis.” Cytokine growth factor Rev 16(2): 179-86.

Moffa, A. B., S. L. Tannheimer, et al. (2004). “Transforming potential of alternatively spliced variants of fibroblast growth factor receptor 2 in human mammary epithelial cells.” Mol Cancer Res 2(11): 643-52.
Palmieri, C., D. Roberts-Clark, et al. (2003). “Fibroblast growth factor 7, secreted by breast fibroblasts, is an interleukin-1 beta-induced paracrine growth factor for human breast cells.” J Endocrinol 177(1): 65-81.
Tamaru, N., Y. Hishikawa, et al. (2004). “Estrogen receptor-associated expression of keratinocyte growth factor and its possible role in the inhibition of apoptosis in human breast cancer.” Lab Invest 84(11): 1460-71.
Watson, C., D. Medina, et al. (1977). “Estrogen receptor characterization in a transplantable mouse mammary tumor.” Cancer Res 37(9): 3344-8.
Zang, X. P., E. C. Bullen, et al. (2006). “Enhanced motility of KGF-transfected breast cancer cells.” Anticancer Res 26(2A): 961-6.
Zang, X. P. and J. T. Pento (2000). “Keratinocyte growth factor-induced motility of breast cancer cells.” Clin Exp Metastasis 18(7): 573-80.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 10 2007 026 877.9, filed Jun. 8, 2007, and U.S. Provisional Application Ser. No. 60/944,153, filed Jun. 15, 2007, are incorporated by reference herein.

The preceding examples can be repeated with similar success by substituting the generically or specifically described reactants and/or operating conditions of this invention for those used in the preceding examples.

1. Use of fibroblast growth factor Fgf7 as biomarker for the progesterone receptor antagonist 11β-(4-acetylphenyl)-17β-hydroxy-17α-(1,1,2,2,2-pentafluoroethyl)-estra-4,9-dien-3-one 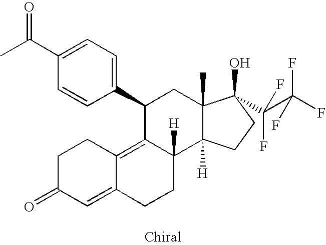
for the manufacture of a medicament for the treatment of cell growth associated with cancer and tumours.

2. Use of the receptor Fgfr2b as biomarker for the progesterone receptor antagonist 11β-(4-acetylphenyl)-17β-hydroxy-17α-(1,1,2,2,2-pentafluoroethyl)-estra-4,9-dien-3-one for the manufacture of a medicament for the treatment of cell growth associated with cancer and tumours.

3. Use of the fibroblast growth factor Fgf7 or of the receptor Fgfr2b as stratifying marker for upregulation of Fgf7 in tumour cells with intrinsic resistance to treatment with 11β-(4-acetylphenyl)-17β-hydroxy-17α-(1,1,2,2,2-pentafluoroethyl)estra-4,9-dien-3-one or of a high Fgfr2 expression in tumour cells for the manufacture of a medicament for the treatment of cell growth associated with cancer and tumours.

4. An in vitro method for determining the activity of the progesterone receptor antagonist 11β-(4-acetylphenyl)-17β-hydroxy-17α-(1,1,2,2,2-pentafluoroethyl)-estra-4,9-dien-3-one in cell cultures and in serum, comprising using fibroblast growth factor Fgf7 or receptor Fgfr2b as biomarker for the determination.

5. A method according to claim 4, wherein the receptor Fgfr2b is used as biomarker for the determination.

6. A method according to claim 4, wherein the fibroblast growth factor Fgf7 or the receptor Fgfr2b is employed as stratifying marker for upregulation of Fgf7 in tumour cells with resistance to treatment with 11β-(4-acetylphenyl)-17β-hydroxy-17α-(1,1,2,2,2-pentafluoroethyl)estra-4,9-dien-3-one or in cells with high Fgfr2 expression.

7. Use according to claim 1, wherein fibroblast growth factor Fgf7 is employed as target for resistance modulation on use of 11β-(4-acetylphenyl)-17β-hydroxy-17α-(1,1,2,2,2-pentafluoroethyl)estra-4,9-dien-3-one.

8. An imaging method for non-invasive determination of FGF7 and FGFR2 in tumour cells with antibodies against these proteins which comprise a label which permit imaging.

9. A method according to claim 8, wherein a fluorescent label or a radioactive label is present as label for the imaging.

10. An in vitro method, comprising using fibroblast growth factor Fgf7 or the receptor Fgfr2b as stratifying marker for upregulation of Fgf7 in tumour cells with resistance to treatment with antiestrogens.

11. An in vitro method according to claim 10, wherein tamoxifen, raloxifene, droloxifen, toremifen, lasofoxifen, arzoxifen, GW5638 *), EM-800 **), idoxifen or basedoxifene is used as antiestrogen.

12. In vitro method for reducing FGF7 expression and inactivating circulating FGF7-blocking antibodies and soluble receptors.

13. A method according to claim 4, wherein the fibroblast growth factor Fgf7 is used as biomarker for the determination.

14. Use according to claim 2, wherein the receptor Fgfr2b is employed as target for resistance modulation on use of 11β-(4-acetylphenyl)-17β-hydroxy-17α-(1,1,2,2,2-pentafluoroethyl)estra-4,9-dien-3-one.
